(19) United States
(12) Patent Application Publication
(10) Pub. No.: US 2004/0047843 A1
(43) Pub. Date: Mar. 11, 2004

(54) Method for spinal cord reconnection

(75) Inventors: Jay M. Meythaler, Birmingham, AL (US); Jean D. Peduzzi-Nelson, Chelsea, AL (US)

Correspondence Address: GIFFORD, KRASS, GROH, SPRINKLE, ANDERSON & CITKOWSKI, PC, 280 N OLD WOODARD AVE, SUITE 400, BIRMINGHAM, MI, 48009, US

(73) Assignee: UAB Research Foundation, Birmingham, AL

(21) Appl. No.: 10/365,021

(22) Filed: Feb. 12, 2003

Related U.S. Application Data
(60) Provisional application No. 60/356,204, filed on Feb. 12, 2002.

(51) Int. Cl. A61K 45/00; C12N 5/08

(52) U.S. Cl. 424/93.7; 435/368

(57) ABSTRACT

According to the present invention, there is disclosed a method for spinal cord reconnection in a damaged spinal cord which includes disposing white matter stem cells or cells capable of developing characteristics of neuronal or supporting cells in an area of spinal cord damage in a subject.

### RELATED APPLICATION

This application claims priority of U.S. Provisional Patent Application Serial No. 60/356,204 filed Feb. 12, 2002, which is incorporated herein by reference.

### TECHNICAL FIELD

The present invention generally relates to the repair of a chronically injured spinal cord. More specifically, the present invention relates to a method for reconnecting a damaged spinal cord by the following methods: 1) implanting white matter stem cells or other cells capable of developing characteristics of neuronal or supporting cells at the location of spinal cord injury in order to reestablish nerve connections at the damaged site; 2) disruption of the scar present at the injury site; 3) increasing the metabolism and/or stimulating growth of cells of the spinal cord; and 4) rehabilitation methods.

### BACKGROUND OF THE INVENTION

In the United States, there are approximately 250,000 chronically paralyzed patients with the number of patients with spinal cord injuries increasing at a rate of approximately 10,000 per year. Currently, there exists no treatment for the restoration of spinal cord function.

Because spinal cord transplantation is neither clinically nor biologically feasible at the present time, other treatments for the restoration of spinal cord function have been suggested. One such mechanism of treatment of chronically injured spinal cords involves the implantation, transplantation, or injection of cells into the body to replace or restore missing spinal cord function, provide a source of growth factors and/or a substrate for growth of axons. These cells can be allowed to proliferate in culture until a sufficient number of cells are produced.

A major drawback of this type of treatment was found to be the lack of a supporting extra-cellular matrix as a tissue framework for tissue expansion and organization into an integrated structure within the damaged spinal cord. When pieces of tissue are not used, transplanted cells in the form of a cell suspension may migrate in the cerebrospinal fluid to other sites in the brain and spinal cord. There is the risk of occluding the ventricular system without the use of a matrix. Several matrix materials are available to provide support for the implanted cells such as methylcellulose, porous hydrogels, and small intestine submucosa. U.S. Pat. No. 5,863,551, incorporated herein by reference, utilizes porous hydrogels to provide the necessary tissue framework through which transplanted cells can proliferate and assemble into functional structures which are capable of restoring spinal cord function. U.S. Pat. No. 6,241,981, incorporated herein by reference, utilizes a derived product from submucosa of the small intestine. However, the prior art methods for repairing damaged spinal cords are deficient in the regeneration and repair of damaged spinal cords.

Accordingly, it would be advantageous and desirable to have a method of treating spinal cord injuries by disposing white matter stem cells or other cells capable of developing neuronal or supporting cells characteristics in an area of spinal cord damage or injury in a subject thereby at least partially restoring spinal cord function and which also overcomes the drawbacks and disadvantages of the prior art. Other cell types that have been used successfully in the lab in combination with a matrix material to treat chronic spinal cord injury include bone stromal cells, olfactory ensheathing cells from the olfactory bulb or the olfactory mucosa, basal cells from the olfactory mucosa. Stem cell-like properties are also found in fat cells and cells from skin and other cells with stem cell-like properties will likely to be found in other parts of the body.

In the case of complete or nearly complete spinal cord injury, disruption of the glial scar that forms at the injury site in the spinal cord. Removal of the glial scar can be done by physical or chemical means. The glial scar is a barrier to the repair process.

It is also advantageous to increase the metabolism in the spinal and/or stimulate growth of cells. There are several ways of increasing metabolism. Chemicals known to increase metabolism include piracetam, cAMP (cyclic adenosine monophosphate), rolipram, caffeine and thyroid hormones. Another method of increasing metabolism is to increase blood flow to a region using a non-thermal pulsed electromagnetic device such as Diapulse. U.S. Pat. No. 6,458,121 incorporated herein by reference, utilizes a non-thermal pulsed electromagnetic field that increases blood flow in the region of application. Growth factors that stimulate growth include IGF-1 (insulin-like growth factor), BDNF (brain derived neurotrophic factor), NT-3 (neurotrophin 3), GDNF (glial derived growth factor).

In order to realize the reparative properties of above said treatments and maximize their effectiveness, rehabilitative methods are necessary. Rehabilitation through the use of an enriched environment was used as an effective treatment in chronic spinal cord injury alone or in combination with other therapies. Other methods used successfully in humans and experimental animals include treadmill therapy, functional electrical stimulation, and swim therapy.

### SUMMARY OF THE INVENTION

According to the present invention, there is disclosed a method for spinal cord reconnection in a damaged spinal cord which includes disposing white matter stem cells or other cells capable of developing neuronal or supporting cells characteristics in an area of spinal cord damage in a subject. If additional cells are needed for transplantation, cells are allowed to multiply in culture.

### DETAILED DESCRIPTION OF THE INVENTION

The present invention provides a method for treating damaged parts of the central nervous system, particularly the spinal cord, but also can include the optic nerve, or peripheral nerves. The method of the present invention includes disposing white matter stem cells or other cells capable of developing neuronal or supporting cells characteristics in an area of spinal cord damage in a subject. The cells are optionally allowed to multiply in culture before transplantation.

The terms “patient” and “subject” mean all animals including humans. Examples of patients or subjects include humans, cows, dogs, cats, goats, sheep, horses, rats, mice and pigs.

Those skilled in the art are easily able to identify patients or subjects having spinal cord injuries including conditions such as partially or completely severed spinal cords.

The method of the present invention includes the isolation or extraction of white matter stem cells or other cells capable of developing neuronal or supporting cells characteristics from the prospective chronic spinal cord injured patient or subject. By utilizing autologous cells, the present method avoids government restrictions on stem cell research and the potential for disease transfer. The white matter stem cells are obtained from extracted white matter or other cells capable of developing neuronal or supporting cells characteristics which is harvested using conventional surgical techniques. Preferably, undifferentiated stem cells are obtained from the peri-ventricular white matter area or other cells capable of developing neuronal or supporting cells characteristics can be obtained from the bone marrow, olfactory bulb, olfactory mucosa, or skin. If insufficient number of cells are obtained, cells will be allowed to multiply in culture.

The extracted and isolated white matter stem cells or other cells capable of developing neuronal or supporting cells characteristics are then combined in an in vitro mixture which includes a hydrogel (neurogel) such as that disclosed in U.S. Pat. No. 5,863,551 of Organogel Canada LTEE or other matrix materials such as submucosa from the small intestine that disclosed in U.S. Pat. No. 6,241,981 or methylcellulose or other substrate. The in vitro mixture can also include other stimulating factors including piracetam, a drug which is known to accelerate nerve cell mitochondrial metabolism or cAMP or thyroid hormones and can also include neurotropic growth factors or other factors that stimulate metabolism and/or encourage growth. These factors that stimulate metabolism and/or encourage growth can be added separately, given locally or systemically and/or at a later time.

After the white matter stem cells or other cells capable of developing neuronal or supporting cells characteristics have been combined with the supporting matrix material, the mixture is then ready to be disposed at the site of spinal cord damage. Prior to disposing the white matter stem cells or other cells capable of developing neuronal or supporting cells characteristics at the site of spinal cord damage, the portion or ends or part (part of the glial scar) of the damaged spinal cord are preferably surgically removed in order to re-induce a minor spinal cord injury and establish the neuronal injury cascade. After the surgical removal of the damaged portion or small part of the glial scar of the spinal cord, the cells are then disposed at the site of spinal cord damage. This is a method to allow the repair process.

Concomitant with or following the disposal of the white matter stem cells or other cells capable of developing neuronal or supporting cells characteristics at the site of injury, macrophage inhibitors or other injury recovering inhibitors, such as CMS 101, are administered to slow down the injury cascade which would otherwise interfere with the hydrogel (neurogel) induced growth. CMS 101 is known to reduce the growth of capillaries in injury areas and may slow down the delivery of macrophages to the injury area and aid in the induced recovery process. U.S. Pat. No. 6,476,001 incorporated herein by reference, utilizes CM101 as a therapy to induce neural repair.

Additionally, electrical stimulation using methods including the “median method” of providing bidirectional electrical current through needles inserted in specific locations in the ears and in appropriate major muscle groups in corresponding locations below the point of injury can also be utilized. The use of electrical stimulation takes advantage of the fact that the neurological system has “borrowed” major nerves to add on small additional peripheral nerves, many of which have a connection in the ear. The net result of this process is to run electrical signals up and down the nerves to stimulate their firing activity. Firing nerves attracts other nerve growth action.

Electrical stimulation is alternatively done by more conventional methods in which electrical current is made to flow from or to a cortical brain region or a region of spinal cord above the site of injury and second from or to a part of the spinal cord distal to injury or in a peripheral nerve connected to the spinal cord distal to the injury.

In addition, the use of the compound 4-aminopyridine (4-AP) can be utilized to provide enhanced signal transmission in a demyelinated (the injury zone) nervous system. The 4-AP may help with signal “jumps” over the remaining gaps as the nerves begin to grow and get closer in proximity to each other.

The neurogel specifically provides three amino acids that act to create a trail for nerve cell axons and dendrites to follow. The electrical stimulation has been shown to stimulate nerve growth and increase the size of dendrite and axon extension. Furthermore, the method of the present invention creates an environment in which stem cells are then given an opportunity to follow electrical stimulation and amino acid trails created by the stimulation and by the natural neuronal injury cascade to promote axon and dendrite growth beyond the point of injury and up and down the spinal cord to the nearest central bodies and their stimulated extension dendrites and axons. These conditions are all designed to create multiple instances for new nerve cells to follow, ideally far past the damage point to form new interconnections.

Additionally, following the initial placement of the white matter stem cells, drugs such as CMS 101 and gamma-amino butyrate can be added to aid in the reconnection/regeneration process. These drugs can be added utilizing a spinal catheter such as that disclosed in PCT Patent Application No. PCT/US00/05740. The use of a wash or bath of the materials found in the neurogel disposed both upstream and downstream from the injury can also be utilized to aid in inducing cell migration and extension.

Other compounds which can be dispensed by the hydrogel/neurogel can include anti-inflammatory substances, cytokine modulators such as steroids, and/or other neuroactive factors.

In view of the teaching presented herein, other modifications and variations of the present invention will readily be apparent to those of skill in the art. The discussion and description are illustrative of some embodiments of the present invention, but are not meant to be limitations on the practice thereof. It is the following claims, including all equivalents, which define the scope of the invention.

Any patents, applications or publications mentioned in the specification are indicative of the levels of those skilled in the art to which the invention pertains. These patents, applications and publications are herein incorporated by reference to the same extent as if each individual publication was specifically and individually indicated to be incorporated by reference.

1. A method for spinal cord reconnection in a damaged spinal cord by disposing white matter stem cells or cells capable of developing characteristics of neuronal or supporting cells in an area of spinal cord damage in a subject.

2. A method according to claim 1 further including the step of obtaining the white matter stem cells or cells capable of developing characteristics of neuronal or supporting cells from the subject prior to said disposing step.

3. A method according to claim 2, wherein the stem cells are undifferentiated.

4. A method according to claim 2 further including the step of combining the stem cells or cells capable of developing characteristics of neuronal or supporting cells with a matrix material.

5. A method according to claim 4, wherein said inducing step comprises contacting the stem cells with at least one nerve cell stimulating agent.

6. A method according to claim 5, wherein the nerve cell stimulating agent comprises a material selected from the group consisting of: a hydrogel, a product of submucosa of small intestine, and methylcellulose.

7. A method according to claim 6, wherein the material comprises a neuroactive agent.

8. A method according to claim 5, wherein the nerve cell stimulating agent comprises piracetam or cAMP.

9. A method according to claim 5, wherein the nerve cell stimulating agent comprises neurotropic factors.

10. A method according to claim 1 further including the step of inducing neuronal injury in the area of spinal cord damage in the subject.

11. A method according to claim 10, wherein said inducing step comprises removing a portion of the damaged spinal cord in order to remove the glial scar.

12. A method according to claim 11, wherein said removing step comprises cutting at least a portion of previously damaged ends from the spinal cord.

13. A method according to claim 12, wherein the stem cells are placed at the site of the induced neuronal injury.

14. A method according to claim 1, wherein said disposing step includes administering at least one immune modulator.

15. A method according to claim 14, wherein the immune modulator inhibits the subject's immune response.

16. A method according to claim 15, wherein the inhibitory immune modulator comprises CMS 101.

17. A method according to claim 1 further including the step of electrically stimulating the stem cells after disposing the stem cells at the site of damage.

18. A method according to claim 1 further including the step of administering 4-aminopyridine to the subject.

19. A method according to claim 1 further including the step of administering gamma aminobutyramide to the subject.

20. A method according to claim 1 further including the step of administering nerve cell stimulating agent is disposed adjacent to the damaged site.
